(19) United States
(12) Patent Application Publication
(10) Pub. No.: US 2007/0281924 A1
(43) Pub. Date: Dec. 6, 2007

(54) MIF inhibitors for treating neuropathic pain and associated syndromes

(76) Inventor: Federico C.A. Gaeta, Mountain View, CA (US)

Correspondence Address: ROBINS & PASTERNAK LLP, 1731 EMBARCADERO ROAD, SUITE 230, PALO ALTO, CA, 94303, US

(21) Appl. No.: 11/807,483

(22) Filed: May 29, 2007

Related U.S. Application Data
(60) Provisional application No. 60/810,034, filed on May 31, 2006.
(60) Provisional application No. 60/812,338, filed on Jun. 8, 2006.
(60) Provisional application No. 60/873,671, filed on Dec. 7, 2006.

(51) Int. Cl. A61K 31/551; A61K 31/496

(52) U.S. Cl. 514/218; 514/253.07; 514/253.08

(57) ABSTRACT

Methods of using MIF inhibitors such as those characterized by structure I are disclosed. The MIF inhibitors can be used for treating conditions such as neuropathic pain and its associated symptoms, as well as for treating drug and behavioral addictions, such as opiate dependence. Additionally, MIF inhibitor compounds can be used for treating withdrawal syndromes after discontinuance of addictive drug use or behavior.

### CROSS REFERENCE TO RELATED APPLICATIONS

This application claims the benefit under 35 U.S.C. §119(e)(1) of provisional application 60/810,034, filed May 31, 2006; provisional application 60/812,338, filed Jun. 8, 2006; and provisional application 60/873,671, filed Dec. 7, 2006, which applications are hereby incorporated by reference in their entireties.

### FIELD OF THE INVENTION

The present invention relates generally to methods of treatment related to the administration of certain MIF inhibitor compounds. In one aspect, the invention relates to methods for treating neuropathic pain. In particular, the present invention pertains to methods of treating or preventing neuropathic pain and its associated symptoms by administration of certain MIF inhibitor compounds. In yet another aspect, the present invention relates generally to methods for treating drug and behavioral addictions. In particular, the present invention pertains to methods for treating addictions, such as opiate dependence, by administration of the MIF inhibitors described herein. Additionally, such MIF inhibitor compounds can be used for treating withdrawal syndromes after discontinuance of addictive drug use or behavior.

### BACKGROUND OF THE INVENTION

In recent years, pain management has become an area of increasing focus in the medical profession, partly due to the growing population of elderly, issues surrounding quality of life, and the growing numbers of patients reportedly suffering from pain. Pain is both a sensory and emotional experience, and is generally associated with tissue damage or inflammation. Typically, pain is divided into two general categories—acute pain and chronic pain. Both differ in their etiology, pathophysiology, diagnosis, and most importantly, treatment.

Acute pain is short term, and is typically of a readily identifiable cause. Patients suffering from acute pain typically respond well to medications. In contrast, chronic pain, medically-defined as pain that lasts for 3-6 months or longer, is often not associated with an obvious injury; indeed, patients can suffer from protracted pain that persists for months or years after the initial insult. While acute pain is generally favorably treated with medications, chronic pain is often much more difficult to treat, generally requiring expert care. Reportedly, according to the American Chronic Pain Association, over 86 million Americans suffer from chronic pain, and the management of chronic pain has long been recognized as an unmet clinical need. Most chronic pain is neuropathic in nature (also referred to as neuralgia). Neuropathic pain can, for instance, manifest itself as burning, stabbing, and shock-like sensations.

Neuropathic pain (NP) is generally thought of as a maladaptive chronic condition in which pain originates from damaged nerves, often yielding pain that is out-of-proportion to the extent of injury. The damage can occur from a physical injury such as trauma or from chemical injury such as chemotherapeutics (e.g., paclitaxol). Neuropathic pain of this type is an important component of a number of syndromes of varying etiologies whose common characteristic is the development of a prolonged and profound pain state. Among these conditions are spinal cord injury, post-herpetic neuralgia, diabetic neuropathy, phantom limb pain, stump/neuroma pain, post-ischemic pain (stroke), fibromyalgia, reflex sympathetic dystrophy (RSD), complex regional pain syndrome (CRPS), cancer-chemotherapeutic induced neuropathic pain, vertebral disk rupture, trigeminal neuralgia, and others.

Unfortunately, neuropathic pain management is at best inconsistent, and often times ineffective. This is in part due to the subjective nature of pain, but also due to poor diagnosis, especially when the chronic pain is not clearly associated with a nerve injury or other insult. Moreover, few, if any, ethical drugs have been prospectively developed for the treatment of chronic pain. Instead, the current medications used to treat chronic pain are “borrowed” from other diseases, most commonly antiepileptic drugs and antidepressants.

Current first-line treatments for chronic pain include opioids, analgesics such as gabapentin, and tricyclic antidepressants. In the instance of opioids, when administered over prolonged periods, undesirable side effects such as drug tolerance, chemical dependency and even physiological addiction can occur. Of treatment regimes currently available for chronic pain, at best, approximately 30% are effective in significantly diminishing the pain, and may lose their efficacy over time. Although numerous pharmacological agents are available for the treatment of neuropathic pain, a definitive therapy has remained elusive.

In instances in which treatment with a single agent proves to be unsuccessful, combination therapy is often then explored as a second line treatment. For example, such combination therapy may employ administration of an opioid agent with an adjuvant analgesic, although the relative doses of each are often subject to prolonged trial and error periods. Oftentimes, triple drug therapy is necessary. Such therapy generally involves a combination of tricyclic antidepressants, anti-convulsants, and a systemic local anesthetic. Patient compliance drops significantly, however, when treatment requires the administration of multiple pharmacologic agents. Recently, researchers reported the use of a combination of morphine and gabapentin in a randomized study for controlling nerve pain (Gilron, I., et al., New Eng. J. of Medicine, Vol 352:1281-82, No. 13, Mar. 31, 2005).

Moreover, it is not only important to consider overall pain relief, but also the type of pain relief. For example, chronic pain is typically viewed as allodynia or hyperalgesia. Allodynia is pain sensation from a stimulus that is not normally painful. The allodynia is typically caused by a physical stimulus and thus referred to as tactile or mechanical allodynia. Hyperalegsia is an exaggerated sensation from a stimulus that is normally painful. The hyperalegsia can occur from a variety of stimuli, but commonly, a patient's reaction to hot and cold stimuli is reported. Importantly, physicians often report that the current drugs are most effective at relieving hyperalgesia although most patients complain from allodynia, particularly mechanical allodynia.

In addition to poor and/or inconsistent efficacy, medications commonly prescribed for neuropathic pain have several other undesirable properties, such as adverse events, duration of action, and complicated dosing and titration regiments.

The most common side-effect of the non-opiate drugs is sedation or somnolence. Based on data from the package inserts for these drugs, as many as 20-30% of patients experience sedation. As mentioned above, the population greatest at risk for chronic pain are elderly. For the elderly, experiencing significant and persistent sedation poses other risks, mainly locomotors function impairment. Such locomotors function impairment can lead to falling and the inability to perform many daily functions such as driving.

The duration of action is also a limitation for most of the leading therapies. This is particularly important as pain, and especially nighttime pain, can lead to depression, insomnia and other factors that impact the patient's overall quality of life. A recent study suggests that patients with chronic pain and concurrent major depression and insomnia report the highest levels of pain-related impairment. This study also found that insomnia in the absence of major depression is also associated with increased pain and distress. (Wilson et al., Clin J Pain 2002 March-April;18(2):77-83.). Therefore, achieving pain relief with a sufficient duration to achieve relief through the night is an important factor for neuropathic pain drugs. Pain-relief drugs such as gabapentin are taken once or more during the night to achieve pain relief—thus disturbing sleep and exacerbating the patient's overall quality of life.

Finally, the dosing or titration of the leading drugs, such as gabapentin, can be complicated. For example, the recommended starting dose for gabapentin in adults with postherpetic neuralgia is a single 300-mg dose on Day 1, 600 mg/day on Day 2 (divided BID), and 900 mg/day on Day 3 (divided TID). If no relief is obtained at these doses, the dose can subsequently be titrated up as needed for pain relief to a daily dose of 1800 mg (divided TID). In clinical studies, efficacy was demonstrated over a range of doses from 1800 mg/day to 3600 mg/day with comparable effects across the dose range.” (Neurontin® Full U.S. Prescribing Information). Other antiepileptic drugs and antidepressants have similar dosing schedules which are similarly complicated, discourage compliance, and increase the chances of incorrect dosing and even overdosing. Further, discontinuing such drugs can also be challenging. For instance, as stated on the Full U.S. Prescribing Information for Neurontin® “. . . [A]s dose is reduced, discontinued or substituted with an alternative medication, this should be done gradually over a minimum of 1 week.”

Turning now to the subject of addiction, the addictiveness of certain drugs and compulsive behaviors is linked to excitation of dopamine mediated reinforcement/reward pathways in the central nervous system (Abbott (2002) Nature 419:872-874; Montague et al. (2004) Nature 431:760-767). Normally dopamine functions to motivate mammals to perform behaviors important for survival, such as eating and sex, but in subjects with addictions, dopamine induces maladaptive behavior. Subjects with addictions feel compelled to use a substance or perform a behavior repeatedly despite experiencing harmful effects. Virtually all drugs of abuse and compulsive behaviors have been shown to increase extracellular dopamine concentrations in the nucleus accumbens of mammals.

Drugs of abuse induce dopamine-mediated dependence characterized by compulsive drug craving and drug seeking behaviors. The World Health Organization (WHO) has classified addictive drugs into nine groups: 1: alcohol, 2. amphetamines, 3. barbiturates, 4. marijuana, 5. cocaine, 6. hallucinogens, 7. khat, 8. opiates, and 9. organic solvents. Dysregulation of dopamine pathways is also associated with compulsive behavioral addictions, such as excessive eating, drinking, smoking, shopping, gambling, sex, and computer use (Comings et al. (2000) Prog. Brain Res. 126:325-341; Comings et al. (1997) 2:44-56; Blum et al. (2000) J. Psychoactive Drugs 32 suppl:i-iv, 1-112; Potenza (2001) Semin. Clin. Neuropsychiatry 6:217-226; Gianoulakis (1998) Alcohol Health Res. World 22:202-210; Bowirrat et al. (2005) Am. J. Med. Genet. B Neuropsychiatr. Genet. 132:29-37; Di Chiara (2005) Physiol. Behav. 86:9-10; Franken et al. (2005) Appetite 45:198-201; Wang et al. (2004) J. Addict Dis. 23:39-53; Aamodt (1998) Nature Med. 4:660; and Koepp et al. (1998) Nature 393:266-268).

In addition, physical and psychological dependence accompanied by withdrawal syndrome is often associated with use of addictive drugs and compulsive behavior. Withdrawal is defined as the appearance of physical and behavioral symptoms upon reduction or cessation of drug use or compulsive behavior. Withdrawal reflects changes occurring in the central nervous system in response to continued use of a substance or repetition of addictive behavior that usurp the normal mechanisms mediating reinforcement and reward of behavior to motivate the addicted individual to continue consuming a drug or repeating compulsive behavior in the face of serious social, legal, physical and professional consequences. Physical symptoms of withdrawal may include intense cravings, irritability, anxiety, dysphoria, restlessness, lack of concentration, lightheadedness, insomnia, tremor, increased hunger and weight gain, yawning, perspiration, lacrimation, rhinorrhoea, dilated pupils, aching of bones, back and muscles, piloerection, hot and cold flashes, nausea, vomiting, diarrhea, weight loss, fever, and increased blood pressure, pulse and respiratory rate.

The management of opioid withdrawal syndrome has long been recognized as an unmet clinical need. Chronic pain afflicts upwards of one in three adults worldwide. Opioid compounds, such as morphine, are frontline therapeutics for the control of chronic pain. Because chronic pain, by definition, persists for many months (and up to the remainder of the patient's life), morphine and like compounds may be given chronically as well. This is a dire problem because opioids induce dependence upon repeated administration, meaning that continuing administration of opioids is required for patients to function normally. When opioids are discontinued, and also during the temporal lag between successive doses of opioids, the patient goes into withdrawal.

Because opioids exert actions in a wide array of brain, spinal cord and bodily tissues, the effects of opioids, and consequent withdrawal symptomologies, are diverse. The signs of withdrawal are generally opposite to the effects of opioids. For example, morphine causes constipation; withdrawal causes diarrhea. Morphine decreases core body temperature, withdrawal raises it. Morphine causes sedation, withdrawal causes agitation. Additional signs of withdrawal include increased pain, dilated pupils, goose pimples, yawning, cramps, muscle aches, restlessness, extreme anxiety, insomnia, nausea and vomiting, sweating, tearing, tachycardia, and increased blood pressure.

Perversely, although pain reduction is the reason that opioids are administered, pain dramatically rebounds during withdrawal such that pain is not only not controlled by the opioids in the area of the original pain complaint, but rather the entire body is now extraordinarily sensitive to touch and temperature stimuli, misinterpreting ordinarily nonpainful stimuli as painful. Light touch becomes painful. Warm and cool become painful. This twist of everyday sensation into threatening pain (along with the other withdrawal symptomology) destroys, on a daily basis, the lives of many millions in the U.S. alone. It creates great suffering in chronic opioid recipients, in patients needing to discontinue opioids, and in recovering drug addicts, whose desire to avoid withdrawal symptoms may prevent them from escaping from illicit drug use.

The problem is compounded by the fact that there is currently no remedy for withdrawal, short of another dose of opioid. As addicts know, another dose of the drug does nothing to solve the problem but instead only masks the problem until the drug yet again wears off. Current approaches to bringing patients and addicts through withdrawal are dire, including “cold turkey”, sedation, and analgesia. “Detoxification” is often induced with naltrexone (an opioid receptor antagonist) under general anaesthesia or benzodiazepine sedation, in a closely monitored environment such as intensive care. Naltrexone induces acute withdrawal, with symptoms that last for about six days. It is only considered for patients in good health. Other currently employed methods to take humans through withdrawal include administration of non-steroidal anti-inflammatory drugs such as paracetamol, anti-emetics such as metoclopramide, anti-diarrheals such as loperamide, diazepam to reduce anxiety and agitation, and clonidine to decrease anxiety, sweating, and changes in heart rate and blood pressure.

In light of the above shortcomings in current approaches for treating chronic pain, there exists a need for improved compositions and methods for treating pain, particularly neuropathic pain and its associated symptoms, and more specifically, neuropathic pain associated with certain conditions such as fibromyalgia, among others. Such approaches should ideally overcome one or more of the problems associated with existing methods for treating chronic pain. Additionally, there remains a need for improved compounds, compositions, and methods of treatment for drug and behavioral addictions. In particular, drugs are needed that attenuate or abolish the dopamine mediated “reward” associated with addicts' cravings and that alleviate symptoms of withdrawal syndromes after discontinuance of drug use or compulsive behavior. The present invention meets these needs.

### SUMMARY OF THE INVENTION

The present invention relates, in one aspect, to a novel approach to treating neuropathic pain, and is based upon the discovery that neuropathic pain can be treated or prevented by administration of a MIF (macrophage migration inhibitory factor) inhibitor compound, and in particular, those having the generalized structure described herein, among others.

The cytokine macrophage migration inhibitory factor (MIF) has been shown to play a role in multiple inflammatory processes, primarily by influencing macrophage function (Bloom and Bennett, Science (1966) 153:80; and Calandra and Roger, Nat. Rev. Immunol. (2003) 3:791-800). Neutralizing antibodies to MIF have been demonstrated to be effective therapeutics in preclinical models of rheumatoid arthritis, endotoxemia and septic shock (Calandra et al., Nat. Med. (2000) 6:164-170; and Santos and Morand, Wein. Med. Wochenschr. (2000) 156:11-18). MIF mRNA is upregulated in microglia three days post-spinal cord injury and may act as a modulator to inflammatory cytokines (Koda et al., Acta Neuropathol. (2004) 108:31-36).

As glial cells (astrocytes, microglia, oligodendrocytes) have cell-type functional similarities to monocytes/macrophages, MIF may influence glial cell activity. Therefore, antagonism of MIF binding to or activity on blood mononuclear and/or glial cells may be central to the herein described efficacy of certain MIF inhibitor compounds in treating neuropathic pain, as well as for treating opiate withdrawal and dependence syndromes.

Thus, MIF antagonists such as those provided herein may represent a new therapeutic approach for the treatment of neuropathic pain, opiate withdrawal and dependence, and for the treatment of other disorders where MIF activity and/or glial activation are implicated. (See, Attorney Docket No.0801-0057 entitled “Method of Antagonizing MIF Activity” filed on even date herewith, for a description of the use of compounds that inhibit MIF for treating neuropathic pain, incorporated herein by reference in its entirety). It is the inventors' belief that systemic administration of the MIF inhibitors described herein is effective in preventing and attenuating, if not eliminating, chronic neuropathic pain, such as that associated with various syndromes. In some instances, administration of a MIF inhibitor can provide an effective treatment for neuropathic pain-related conditions that are non-responsive to existing therapies.

Accordingly, in one aspect, the invention provides a method of treating a mammalian subject suffering from neuropathic pain by administering to the subject a therapeutically effective amount of a MIF inhibitor having the following structure: 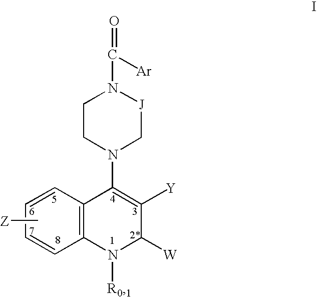
including stereoisomers, prodrugs, and pharmaceutically acceptable salts thereof.

In structure I, the variables typically correspond to the following: Ar is selected from aryl, substituted aryl, heteroaryl, and substituted heteroaryl; J is lower alkylene or carbonyl (C═O); Y is selected from —NO₂, CO₂R₂; R₀ indicates the absence of a substituent on N1; R₁ indicates the presence of a substituent on N1 selected from hydrogen, alkyl, —(CH₂)ₙAr, and (CH₂)ₙN(CH₃)₂ where n ranges from 1-5; R₂ is lower alkyl; Z is selected from —H, halo, and lower alkyl; and, when N₁ is attached to R₁, W, when taken together with C₂ (indicated by an “*”), forms a carbonyl (=O), and when N₁ is attached to R₀, then N₁ forms a double bond with C₂, and W is either —OR₂ or —SR₂. As a result of administering a compound corresponding to structure I above, the subject experiences relief of neuropathic pain.

In reference to structure I shown above, particular embodiments of the invention include the following, among others. For example, in one embodiment, Ar is selected from thiophene and pyrrole.

In yet another embodiment, Z is positioned at the 6-position of the quinoline or 2-quinolone ring structure.

In yet another embodiment, Z is selected from hydrogen, chloro, and methyl.

In yet another particular embodiment, W, when taken together with C₂, forms a carbonyl.

In yet an alternative embodiment, W is either —OR₂ or —SR₂, and N₁ forms a double bond with C₂.

Illustrative MIF structures for use in the methods provided herein include the following:

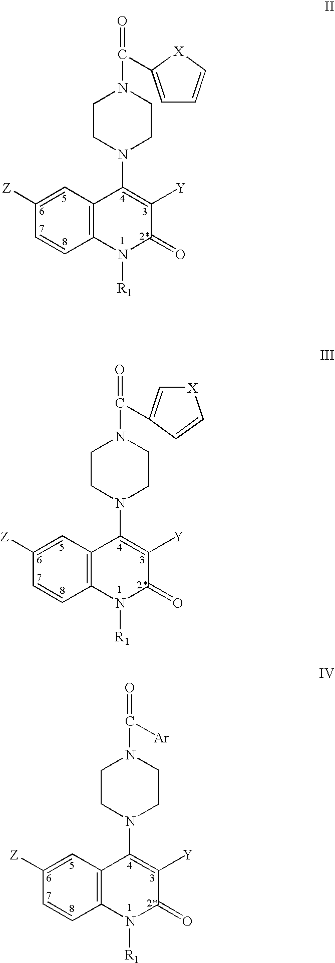

where the variables have the values previously described, and X is sulfur or oxygen.

In a preferred embodiment, any one of structures II, III, or IV is one where Z is selected from hydrogen, chloro, and methyl; and Y is selected from —NO₂, —C(O)OCH₃, —C(O)OCH₂CH₃, —C(O)OCH₂CH₂CH₃, and —C(O)OCH(CH₃)₂.

Yet additional exemplary MIF structures for use in the methods provided herein include the following:

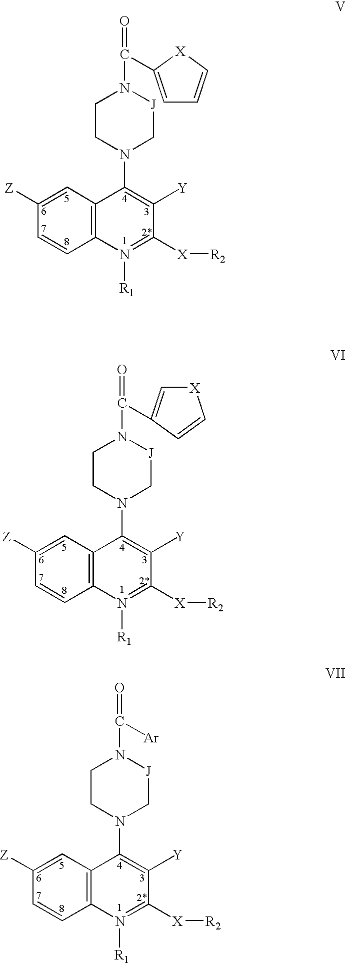

where the variables have the values previously described, and X is sulfur or oxygen.

In a preferred embodiment, any one of structures V, VI or VII is one where Z is selected from hydrogen, chloro, and methyl; and Y is selected from —NO₂, —C(O)OCH₃, —C(O)OCH₂CH₃, —C(O)OCH₂CH₂CH₃, and —C(O)OCH(CH₃)₂.

Mammalian subjects suitable for treatment using a MIF inhibitor as described herein include those suffering from postherpetic neuralgia, trigeminal neuralgia, and neuropathic pain associated with a condition selected from the group consisting of herpes, HIV, traumatic nerve injury, stroke, post-ischemia, fibromyalgia, reflex sympathetic dystrophy, complex regional pain syndrome, and cancer-chemotherapeutic-induced neuropathic pain.

A therapeutic dosage amount may be achieved by intermittent administration, or administration once daily (i.e., in a single dose), twice daily (i.e., in two separate doses), three times daily, or may be administered as multiple doses over a time course of several days, weeks, or even months. Such administering is typically over a duration of time effective to result in a diminution, and ideally elimination or even reversal, of neuropathic pain. Exemplary durations of treatment include at least about one week, from 1 week to 1 month, from two weeks to 2 months, up to about 6 months, up to about 12 months or even longer. In one particular embodiment, treatment lasts from about 1 week to about 50 weeks.

In a preferred embodiment of the treatment method, the administering is over a duration of time effective to result in elimination of the neuropathic pain.

In a further embodiment of the method, a MIF inhibitor of the invention is administered in combination with at least one other agent effective for treating pain. Such agents include ibudilast, gabapentin, memantine, pregabalin, morphine and related opiates, cannabinoids, tramadol, lamotrigine, carbamazepine, duloxetine, milnacipran, and tricyclic antidepressants, among others. (See, US Patent Publication No. 2006/0160843, published Jul. 20, 2006, for a description of the use of ibudilast to treat neuropathic pain, incorporated herein by reference in its entirety).

In yet another embodiment, a MIF inhibitor described by structure I, when administered either singly or as part of a combination therapy, is administered either systemically or centrally (e.g., by intrathecal administration, i.e., into the cerebrospinal fluid surrounding the spinal cord).

According to yet a further embodiment, the MIF inhibitor is administered systemically, e.g. via intravenous, subcutaneous, oral, intranasal, sublingual or other systemic routes, to a mammalian, e.g., human, subject for the treatment of neuropathic pain, e.g., a neuropathic pain syndrome.

In another aspect, the invention provides a composition or combination effective for treating neuropathic pain. The composition comprises a combination of: (i) a MIF inhibitor as described herein, and (ii) at least one additional agent effective for treating neuropathic pain, where each of the components is either contained in a single composition or dosage form (such as in an admixture), or is present as a discrete or separate entity (e.g., in a kit).

A composition of the invention may optionally include one or more pharmaceutically acceptable excipients.

In yet another aspect, the invention encompasses a kit comprising a MIF inhibitor of the type described herein, for the treatment of neuropathic pain or a related syndrome, and optionally, at least one additional agent effective for treating neuropathic pain, for simultaneous, sequential or separate use.

In yet another aspect, the invention provides a method for suppressing the release of dopamine in the nucleus accumbens of a subject comprising administering to the subject an effective amount of a MIF inhibitor compound having a generalized structure as described above.

In certain embodiments, the subject is suffering from an addiction. The addiction may, in certain instances, be a drug addiction, for example, an opiate, cocaine, amphetamine, methamphetamine, cannabinoid, alcohol, or nicotine addiction. Alternatively, the addiction may be a behavioral addiction, for example, an eating, drinking, smoking, shopping, gambling, sex, or computer use addiction.

In another aspect, the invention provides a method for treating an addiction such as those described above by administering to a subject in need thereof a therapeutically effective amount of a MIF inhibitor as described herein.

Administration of a MIF inhibitor as described herein may, in certain embodiments, be used for one or more of the following: (i) for diminishing or eliminating addiction-related behavior of a subject, (ii) for diminishing or eliminating cravings associated with addiction to a drug in a subject, (iii) for diminishing or eliminating tolerance to a drug in a subject, and/or (iv) for diminishing or eliminating the incentive salience of drug- or addictive behavior-associated cues in a subject.

In yet another embodiment, provided herein is a method for diminishing or eliminating symptoms of withdrawal syndrome in a subject by administration of a MIF inhibitor as described herein.

Each of the herein-described features of the invention is meant to apply equally to each and every embodiment as described herein, unless otherwise indicated.

Additional objects, advantages and novel features of the invention will be set forth in the description that follows, and in part, will become apparent to those skilled in the art upon reading the following, or may be learned by practice of the invention.

### DETAILED DESCRIPTION OF THE INVENTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed.

All publications, patents and patent applications cited herein, whether supra or infra, are hereby incorporated by reference in their entirety.

Definitions

Before describing the present invention in detail, it is to be understood that this invention is not limited to particular administration modes, patient populations, and the like, as such may vary, as will be apparent from the accompanying description and figures.

It must be noted that, as used in this specification and the intended claims, the singular forms “a,” “an,” and “the” include plural referents unless the context clearly dictates otherwise. Thus, for example, reference to “a drug ” includes a single drug as well as two or more of the same or different drugs, reference to “an optional excipient” refers to a single optional excipient as well as two or more of the same or different optional excipients, and the like.

In describing and claiming the present invention, the following terminology will be used in accordance with the definitions described below. The following definitions are meant to apply regardless of whether a term is used by itself or in combination with another term. That is to say, the definition of “alkyl” applies to “alkyl” as well as to the “alkyl” portions of “alkoxy”, “alkylamino”, alkylene, etc.

“Alkyl” refers to a hydrocarbon chain, typically ranging from about 1 to 20 atoms in length. Such hydrocarbon chains are preferably saturated and may be branched or straight chain, although typically straight chain is preferred. Exemplary alkyl groups include methyl, ethyl, propyl, isopropyl, butyl, pentyl, 1-methylbutyl, 1-ethylpropyl, 3-methylpentyl, and the like. As used herein, “alkyl” includes cycloalkyl when three or more carbon atoms are referenced.

“Lower” in reference to a particular functional group means a group having from 1-6 carbon atoms.

For example, “lower alkyl” refers to an alkyl group containing from 1 to 6 carbon atoms, and may be straight chain or branched, as exemplified by methyl, ethyl, n-propyl, isopropyl, 1-ethylpropyl, 1,2-dimethylpropyl, n-butyl, i-butyl, sec-butyl, t-butyl, and the like.

“Non-interfering substituents” are those groups that, when present in a molecule, are typically non-reactive with other functional groups contained within that molecule.

The term “substituted” as in, for example, “substituted alkyl” or “substituted aryl” or “substituted heteroaryl” as used herein is a broad term and is used in its ordinary sense, without limitation, to refer to any functional group that replaces at least one hydrogen atom in a given reference moiety. For example, a substituted alkyl moiety is one in which at least one of the hydrogens of the alkyl moiety is replaced with a non-interfering substituent. Substituents (such as those “substituted” on a given reference moiety) include but are not limited to: C₃-C₈ cycloalkyl (e.g., cyclopropyl, cyclobutyl, and the like), halogen, (e.g., fluoro, chloro, bromo, and iodo), cyano, oxo, acyl, ester, sulfhydryl, amino, thioalkyl, carbonyl, carboxyl, carboxamido, alkoxy, lower alkyl, aryl, substituted aryl, phenyl, substituted phenyl, cyclic amides (e.g., cyclopentamide, cyclohexamide, etc., morpholinamide, tetrahydroquinolineamide, tetrahydroisoquinolineamide, coumarinamides, and the like). For substitutions on a phenyl ring, the substituents may be in any orientation (i.e., ortho, meta, or para). Preferred substituents include halogens, and lower alkoxy groups.

“Alkoxy” refers to an —O—R group, wherein R is alkyl or substituted alkyl, preferably C₁-C₂₀ alkyl (e.g., methoxy, ethoxy, propyloxy, benzyl, etc.), preferably C₁-C₇.

As used herein, “alkenyl” refers to a branched or unbranched hydrocarbon group of 1 to 15 atoms in length, containing at least one double bond, such as ethenyl, n-propenyl, isopropenyl, n-butenyl, isobutenyl, octenyl, decenyl, tetradecenyl, and the like.

The term “alkynyl” as used herein refers to a branched or unbranched hydrocarbon group of 2 to 15 atoms in length, containing at least one triple bond, ethynyl, n-propynyl, isopropynyl, n-butynyl, isobutynyl, octynyl, decynyl, and so forth.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. Preferred aryl groups contain one or two aromatic rings. Examples include phenyl, benzyl, naphthyl, and the like.

“Heteroaryl” is an aryl group containing from one to four heteroatoms, preferably N, O, or S, or a combination thereof. Heteroaryl rings may also be fused with one or more cyclic hydrocarbon, heterocyclic, aryl, or heteroaryl rings. Exemplary heteroaryl rings include pyridine, pyridazine, pyrrole, pyrazole, triazole, imidazole, oxazole, isoxazole, thiazole, isothiazole, thiophene, tetrahydroisoquinoline, tetrahydroisoquinolineamide, coumarin, courmarinamide, and the like.

“Heterocycle” or “heterocyclic” means one or more rings of 5-12 atoms, preferably 5-7 atoms, with or without unsaturation or aromatic character and having at least one ring atom which is not a carbon. Preferred heteroatoms include sulfur, oxygen, and nitrogen.

“Substituted heteroaryl” is heteroaryl having one or more non-interfering groups as substituents.

“Amino” as used herein, encompasses both mono-substituted amino and di-substituted amino compounds. For example, amino refers to the moiety, —NRaRb, where Ra and Rb are each independently —H, alkyl, aryl, or alkylaryl.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

By “pathological pain” is meant any pain resulting from a pathology, such as from functional disturbances and/or pathological changes, lesions, bums and the like. One form of pathological pain is “neuropathic pain” which is pain thought to initially result from nerve damage but extended or exacerbated by other mechanisms including glial cell activation. Examples of pathological pain include, but are not limited to, thermal or mechanical hyperalgesia, thermal or mechanical allodynia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, and the like.

“Hyperalgesia” means an abnormally increased pain sense, such as pain that results from an excessive sensitiveness or sensitivity. Examples of hyperalgesia include but are not limited to cold or heat hyperalgesia.

“Hypalgesia” (or “hypoalgesia”) means the decreased pain sense.

“Allodynia” means pain that results from normally non-noxious stimulus to the skin or body surface. Examples of allodynia include, but are not limited to, cold or heat allodynia, tactile or mechanical allodynia, and the like.

“Nociception” is defined herein as pain sense. “Nociceptor” herein refers to a structure that mediates nociception. The nociception may be the result of a physical stimulus, such as, mechanical, electrical, thermal, or a chemical stimulus. Nociceptors are present in virtually all tissues of the body.

“Analgesia” is defined herein as the relief of pain without the loss of consciousness. An “analgesic” is an agent or drug useful for relieving pain, again, without the loss of consciousness.

The term “central nervous system” or “CNS” includes all cells and tissue of the brain and spinal cord of a vertebrate. Thus, the term includes, but is not limited to, neuronal cells, glial cells, astrocytes, cerebrospinal fluid (CSF), interstitial spaces and the like.

“Glial cells” refer to various cells of the CNS also known as microglia, astrocytes, and oligodendrocytes.

The term “addiction” is defined herein as compulsively using a drug or performing a behavior repeatedly that increases extracellular dopamine concentrations in the nucleus accumbens. An addiction may be to a drug including, but not limited to, psychostimulants, narcotic analgesics, alcohols and addictive alkaloids such as nicotine, cannabinoids, or combinations thereof. Exemplary psychostimulants include, but are not limited to, amphetamine, dextroamphetamine, methamphetamine, phenmetrazine, diethylpropion, methylphenidate, cocaine, phencyclidine, methylenedioxymethamphetamine and pharmaceutically acceptable salts thereof. Exemplary narcotic analgesics include, but are not limited to, alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine and pharmaceutically acceptable salts thereof. Addictive drugs also include central nervous system depressants, such as barbiturates, chlordiazepoxide, and alcohols, such as ethanol, methanol, and isopropyl alcohol. The term addiction also includes behavioral addictions, for example, compulsive eating, drinking, smoking, shopping, gambling, sex, and computer use.

A subject suffering from an addiction experiences addiction-related behavior, cravings to use a substance in the case of a drug addiction or overwhelming urges to repeat a behavior in the case of a behavioral addiction, the inability to stop drug use or compulsive behavior in spite of undesired consequences (e.g., negative impacts on health, personal relationships, and finances, unemployment, or imprisonment), reward/incentive effects associated with dopamine release, salience of drug- or behavior-associated cues, dependency, tolerance, or any combination thereof.

Addiction-related behavior in reference to a drug addiction includes behavior resulting from compulsive use of a drug characterized by dependency on the substance. Symptomatic of the behavior is (i) overwhelming involvement with the use of the drug, (ii) the securing of its supply, and (iii) a high probability of relapse after withdrawal.

The terms “subject”, “individual” or “patient” are used interchangeably herein and refer to a vertebrate, preferably a mammal. Mammals include, but are not limited to, murines, rodents, simians, humans, farm animals, sport animals and pets.

The terms “pharmacologically effective amount” or “therapeutically effective amount” of a composition or agent, as provided herein, refer to a nontoxic but sufficient amount of the composition or agent to provide the desired response, such as a reduction or reversal of neuropathic pain or suppression of the release of dopamine in the nucleus accumbens of a subject. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein. In the case of an effective amount of a composition or agent administered for treatment of a drug or behavioral addition, such is an amount that brings about a positive therapeutic response in treatment of a drug or behavioral addiction, such as diminishing or eliminating addiction-related behavior of a subject, diminishing or eliminating cravings associated with addiction to a drug or a behavior in a subject, diminishing or eliminating tolerance to a drug in a subject, diminishing or eliminating the incentive salience of drug- or behavior-associated cues in a subject, and/or diminishing or eliminating symptoms of withdrawal caused by reduction or cessation of addictive drug use or behavior by a subject.

The term “about”, particularly in reference to a given quantity, is meant to encompass deviations of plus or minus five percent.

“Treatment” or “treating” neuropathic pain includes: (1) preventing pain, i.e. causing pain not to develop or to occur with less intensity in a subject that may be exposed to or predisposed to pain but does not yet experience or display pain, (2) inhibiting pain, i.e., arresting the development or reversing pain, or (3) relieving pain, i.e., decreasing the amount of pain experienced by the subject.

By “treating existing pain” is meant attenuating, relieving or reversing neuropathic pain in a subject that has been experiencing pain for at least 24 hours, such as for 24-96 hours or more, such as 25 . . . 30 . . . 35 . . . 40 . . . 45 . . . 48 . . . 50 . . . 55 . . . 65 . . . 72 . . . 80 . . . 90 . . . 96 . . . 100, etc. hours. The term also intends treating pain that has been occurring long-term, such as for weeks, months or even years.

Methods for Treating Neuropathic Pain

As described previously, the inventors have discovered the MIF inhibitors described generally by structure I to be effective in the treatment of neuropathic pain, e.g., neuropathic pain associated with certain syndromes such as viral neuralgias (e.g., herpes, AIDS), diabetic neuropathy, phantom limb pain, stump/neuroma pain, post-ischemic pain (stroke), fibromyalgia, reflex sympathetic dystrophy (RSD), complex regional pain syndrome (CRPS), cancer pain, vertebral disk rupture, and trigeminal neuralgia, cancer-chemotherapy-induced neuropathic pain, among others. Thus, using standard pain models as described herein, it can be demonstrated that administration of a MIF inhibitor, e.g., corresponding generally to structure I, is surprisingly effective in providing a measurable reduction in the severity of neuropathic pain, and in particular, in providing a measurable reduction in the severity if not reversal of certain types of neuropathic pain such as mechanical allodynia. Additional features of the invention are described herein.

Quinoline or 2-Quinolone Based MIF Inhibitors

The methods of the invention for the treatment of neuropathic pain are based upon administration of a MIF inhibitor having the general structure shown below. 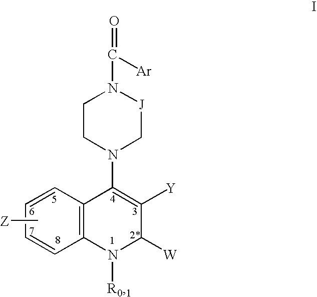
In the above structure, the numbered ring system may correspond to either a quinoline or a quinolone. For instance, when N₁ is attached to R₁ (that is to say, when the ring nitrogen N1 is trivalent, and a single bond connects N1 and C2 (indicated in the structure above by an asterisk), then W, when taken together with C₂, forms a carbonyl (=O) such that the fused aromatic two ring system shown above corresponds to a quinolone. Alternatively, when N₁ is attached to R₀ (indicating the absence of a substituent or functional group attached to N1), then N₁ forms a double bond with C₂, such that the fused aromatic two ring system shown above corresponds to a quinoline, where W is either —OR₂ or —SR₂, and R₂ is lower alkyl. For example, W may be —OCH₃, —OCH₂CH₃, —OCH₂CH₂CH₃, —OCH(CH₃)₂, —SCH₃, —SCH₂CH₃, —SCH₂CH₂CH₃, —SCH(CH₃)₂, among others.

In the instance where the numbered ring system is a quinolone, then R₁, the substituent covalently attached to N1, is typically selected from hydrogen (meaning that N1 is unsubstituted), alkyl (preferably lower alkyl), alkylaryl, alkyl substituted aryl, alkylamino, alkyl N-substituted amino, alkyl N, N-disubstituted amino, where the amino group is typically substituted with one or two lower alkyl groups. A preferred substituted amino is methyl amino or dimethylamino. Representative R₁ groups include —CH₃, —(CH₂)ₙAr, and (CH₂)ₙN(CH₃)₂ where n ranges from 1-5 and Ar is as defined elsewhere herein (i.e., is selected from aryl, substituted aryl, heteroaryl, and substituted heteroaryl).

In reference to structure I, “Ar”, the group covalently attached to the carbonyl carbon, is selected from aryl, substituted aryl, heteroaryl, and substituted heteroaryl. Preferred aryl groups include phenyl, while preferred substituted aryl groups include substituted phenyl. Exemplary substituted phenyl groups include a phenyl substituted with one or more halogens (e.g., chloro, iodo, or fluoro), or with one or more alkoxy groups such as methoxy or ethoxy. The substituents on the phenyl ring may be in any orientation. For example, for a mono-substituted phenyl ring (i.e., a phenyl ring having one substituent in addition to the carbonyl carbon to which it is directly covalently attached), the substituents may be ortho, meta, or para. For a di-substituted phenyl ring, the substituents, including the carbonyl, may, e.g., form a 1,2,3-substituted phenyl ring, or a 1,2,4-substituted phenyl ring, or a 1,3,5-substituted phenyl ring.

Preferred heteroaryl groups include pyrrole and thiophene, where the squiggly line below indicates the point of attachment to the piperazine ring, and X is either oxygen or sulfur.

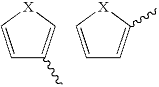

In reference to the “piperazine ring”, although this ring system is referred to herein as a piperazine, such terminology is truly proper only in the instance where J is a methylene group, —CH₂—. However, for convenience sake, in reference to generalized structure I, the six membered ring having the two opposing nitrogen atoms is referred to as a piperazine, although J may be a lower alkylene or a carbonyl (C═O). In instances in which J is a lower alkylene that is not a methylene, the ring will contain more than six ring atoms, e.g., 7, 8, 9, 10, 11, 12, etc. Preferably, J is methylene or carbonyl.

Turning now to the quinoline or 2-quinolone ring system, carbon-3 possesses a substituent, Y. Values for Y include —NO₂ and —CO₂R₂, where R₂ is lower alkyl. For instance, illustrative substituents at C3 include —C(O)OCH₃, —C(O)OCH₂CH₃, —C(O)OCH₂CH₂CH₃, —C(O)OCH(CH₃)₂, and the like.

Finally, the quinoline or quinolone ring system optionally contains a substituent, Z.

Values for Z include halo and lower alkyl. Particularly preferred Z substituents include chloro and methyl.

Illustrative MIF structures for use in the methods provided herein include the following: 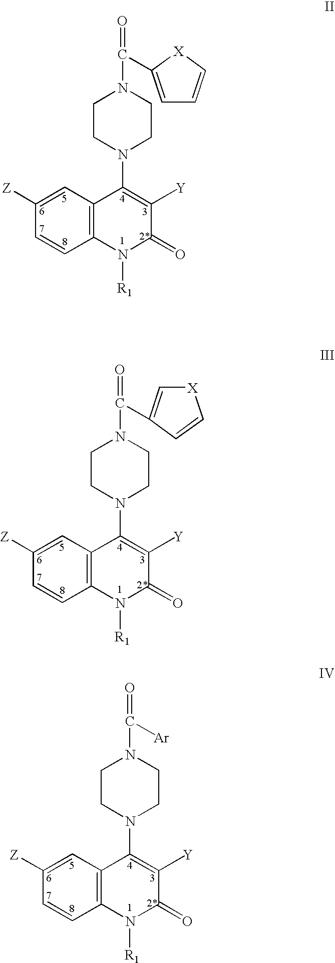
where the variables indicated in the structure above have the values previously described, and X is sulfur or oxygen.

In a preferred embodiment, any one of structures II, III, or IV is one where Z is selected from hydrogen, chloro, and methyl; and Y is selected from —NO₂, —C(O)OCH₃, —C(O)OCH₂CH₃, —C(O)OCH₂CH₂CH₃, and —C(O)OCH(CH₃)₂.

Yet additional exemplary MIF structures for use in the methods provided herein include the following:

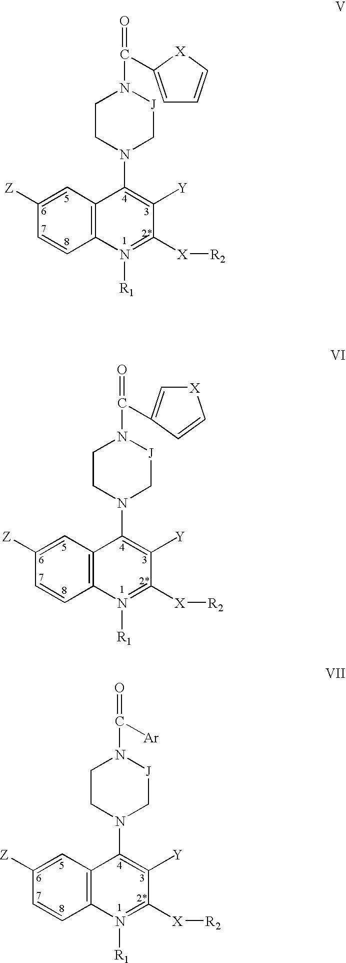

where the variables have the values previously described, and X is sulfur or oxygen.

In a preferred embodiment, any one of structures V, VI or VII is one where Z is selected from hydrogen, chloro, and methyl; and Y is selected from —NO₂, —C(O)OCH₃, —C(O)OCH₂CH₃, —C(O)OCH₂CH₂CH₃, and —C(O)OCH(CH₃)₂.

Additional MIF inhibitors for use in the invention include those described in U.S. Patent Publication No. 2003/0195194, incorporated herein by reference in its entirety, with particular direction to sections therein describing chemical structures of MIF inhibitors and their methods of synthesis.

The MIF inhibitors of the present invention can be synthesized using conventional methods of organic synthesis. See, for example, J. March, Advanced Organic Chemistry: Reactions Mechanisms and Structure, 4th Ed. (New York: Wiley-Interscience, 1992), and Comprehensive Organic Functional Group Transformations II, Volumes 1-7, Second Ed.: A Comprehensive Review of the Synthetic Literature 1995-2003 (Organic Chemistry Series), Eds. Katritzky, A. R., et al., Elsevier Science. Additionally, synthetic methodologies useful for forming the MIF inhibitors described herein may be found in U.S. Patent Publication No. 2003/0195194, ibid.

As stated previously, a reference to any one or more of the herein-described drugs, in particular, a MIF inhibitor corresponding to structure I, is meant to encompass, where applicable, any and all enantiomers, mixtures of enantiomers including racemic mixtures, prodrugs, pharmaceutically acceptable salt forms, hydrates (e.g., monohydrates, dihydrates, etc.), solvates, different physical forms (e.g., crystalline solids, amorphous solids), metabolites, and the like. Where applicable, the MIF inhibitors provided herein may be employed in their free base form, or alternatively, may be in the form of an acid addition salt. Acid addition salts of the free base form of certain amino compounds include those formed from both organic and inorganic acids. Suitable organic acids include maleic, fiunaric, benzoic, ascorbic, succinic, methanesulfonic, acetic, oxalic, propionic, tartaric, salicyclic, citric, and the like. Suitable inorganic acids include hydrochloric, sulfuric, nitric, and the like.

Method of Administration

As set forth above, the present invention encompasses a method of treating a mammalian subject suffering from neuropathic pain by administering a therapeutically effective dosage of a MIF inhibitor corresponding to structure I. Such administering is effective to decrease the amount of neuropathic pain experienced by the subject, i.e., to result in a significant attenuation or even reversal of neuropathic pain.

Therapeutic amounts can be empirically determined and will vary with the particular condition being treated, the subject, the specific structure of the MIF inhibitor, and the particular efficacy and toxicity of each of the active agents contained in the composition. The actual dose to be administered will vary depending upon the age, weight, and general condition of the subject as well as the severity of the condition being treated, the judgment of the health care professional, and particular mode of administration.

The method of the invention may, in certain instances, comprise a step of selecting a subject experiencing neuropathic pain prior to administering thereto a MIF inhibitor of the invention. Such subjects are typically selected from those suffering from postherpetic neuralgia, trigeminal neuralgia, and neuropathic pain associated with a condition selected from the group consisting of herpes, HIV, traumatic nerve injury, stroke, post-ischemia, fibromyalgia, reflex sympathetic dystrophy, complex regional pain syndrome, spinal cord injury, phantom limb pain, multiple-sclerosis, sciatica, and cancer or cancer-chemotherapeutic-induced neuropathic pain.

The method of the invention may be effective to not only significantly attenuate neuropathic pain, for example, mechanical allodynia, but to even reverse it, such that the resulting pain relief is long-lasting. Thus, the administering of a MIF inhibitor as described herein may be effective to result in sustained attenuation of neuropathic pain for an overnight duration. For example, a therapeutically effective dose of a MIF inhibitor of the invention may be effective to treat neuropathic pain for a duration of up to at least 4 hours, 6 hours, 8 hours, 10 hours, 12 hours, 16 hours, 18 hours or even 20 hours or greater.

A MIF inhibitor of the invention may also be administered in combination with an additional agent effective for treating neuropathic pain. Exemplary agents include ibudilast, gabapentin, memantine, pregabalin, morphine and related opiates, cannabinoids, tramadol, lamotrigine, lidocaine, carbamazepine, duloxetine, milnacipran, and tricyclic antidepressants.

Administration of a MIF inhibitor of the invention may also be effective in not only attenuating cancer-chemotherapeutic agent-induced neuropathy, but can also prevent the development of such neuropathy. Examples of chemotherapeutic agents known to result in patient neuropathy include taxol, vinblastine, and vincristine. Thus, administration of a MIF inhibitor of the types described herein may be effective in attenuating or reversing neuropathic pain associated with the administration of such agents for the treatment of cancer.

The method of the invention may also offer an additional advantage over existing neuropathic pain therapies, since existing neuropathic pain medications have sedation as a major side-effect, while the MIF inhibitors provided herein may not.

Preferred methods of delivery of MIF inhibitor-based therapeutic formulations for the treatment of neuropathic pain include systemic and localized delivery, i.e., directly into the central nervous system. Such routes of administration include but are not limited to, oral, intra-arterial, intrathecal, intraspinal, intramuscular, intraperitoneal, intravenous, intranasal, and inhalation routes.

More particularly, a MIF inhibitor-based formulation of the present invention may be administered for therapy by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular neuralgia-associated syndrome being treated, and the specific MIF inhibitor employed.

One preferred mode of administration for delivery of a MIF inhibitor is directly to neural tissue such as peripheral nerves, the retina, dorsal root ganglia, neuromuscular junction, as well as the CNS, e.g., to target spinal cord glial cells by injection into, e.g., the ventricular region, as well as to the striatum (e.g., the caudate nucleus or putamen of the striatum), spinal cord and neuromuscular junction, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

A particularly preferred method for targeting spinal cord glia is by intrathecal delivery, rather than into the cord tissue itself.

Another preferred method for administering the MIF inhibitor compositions of the invention is by delivery to dorsal root ganglia (DRG) neurons, e.g., by injection into the epidural space with subsequent diffusion to DRG. For example, a MIF inhibitor composition as provided herein can be delivered via intrathecal cannulation. See, e.g., Chiang et al., Acta Anaesthesiol. Sin. (2000) 38:31-36; Jain, K. K., Expert Opin. Investig. Drugs (2000) 9:2403-2410.

Yet another mode of administration to the CNS uses a convection-enhanced delivery (CED) system. In this way, a MIF inhibitor can be delivered to many cells over large areas of the CNS. Any convection-enhanced delivery device may be appropriate for delivery of a MIF inhibitor of structure I. In a preferred embodiment, the device is an osmotic pump or an infusion pump. Both osmotic and infusion pumps are commercially available from a variety of suppliers such as Durect Corporation (suppliers of Alzet® osmotic pumps, Cupertino, Calif.) and Alza, Inc., Palo Alto, Calif. Typically, a MIF inhibitor-based composition of the invention is delivered via a CED device as follows. A catheter, cannula or other injection device is inserted into CNS tissue in the chosen subject. Stereotactic maps and positioning devices are available, for example from ASI Instruments, Warren, Mich. Positioning may also be conducted by using anatomical maps obtained by CT and/or MRI imaging to help guide the injection device to the chosen target. For a detailed description regarding CED delivery, see, e.g., U.S. Pat. No. 6,309,634, incorporated herein by reference in its entirety.

A MIF inhibitor composition of the invention, when comprising more than one active agent, may be administered as a single combination composition comprising a combination of a MIF inhibitor and at least one additional active agent effective in the treatment of neuropathic pain. In terms of patient compliance and ease of administration, such an approach is preferred, since patients are often adverse to taking multiple pills or dosage forms, often multiple times daily, over the duration of treatment. Alternatively, albeit less preferably, the combination of the invention is administered as separate dosage forms. In instances in which the drugs comprising the therapeutic composition of the invention are administered as separate dosage forms and co-administration is required, the MIF inhibitor and each of the additional active agents may be administered simultaneously, sequentially in any order, or separately.

Dosages

Therapeutic amounts can be empirically determined by those skilled in the art and will be adjusted to the requirements of each particular case. That is to say, the therapeutic amount may vary with the particular condition being treated, the subject, the structure of the MIF inhibitor employed, and the efficacy and toxicity of each of the active agents contained in the composition if more than one active agent is present. The actual dose to be administered will vary depending upon the age, weight, and general condition of the subject as well as the severity of the condition being treated, the judgment of the health care professional, and particular MIF inhibitor being administered.

Generally, a therapeutically effective amount of a MIF inhibitor as provided herein will range from a total daily dosage of about 0.1 and 200 mg/day, more preferably, in an amount between 1-200 mg/day, 30-200 mg/day, 1-100 mg/day, 30-100 mg/day, 30-200 mg/day, 1-60 mg/day, 1-40 mg/day, or 1-10 mg/day, administered as either a single dosage or as multiple dosages. Depending upon the dosage amount and precise condition to be treated, administration can be one, two, or three times daily, or even more, for a time course of one day to several days, weeks, months, and even years, and may even be for the life of the patient. Intermittent dosing may also be employed, e.g., in response to neuropathic pain, with a maximal dose not to be exceeded as recommended by the practicing physician. Illustrative dosing regimes will last a period of at least about a week, from about 1-4 weeks, from 1-3 months, from 1-6 months, from 1-50 weeks, from 1-12 months, or longer.

Pain Models

The ability of a MIF inhibitor to treat neuropathic pain can be evaluated by any of the standard pain models known in the art. Examples of such models are as follows.

Carrageenan-induced Paw Hyperalgesia Model: The carrageenan paw hyperalgesia test is a model of inflammatory pain. A subcutaneous injection of carrageenan is made into the left hindpaws of rats. The rats are treated with a selected agent, e.g., a MIF inhibitor, before, e.g., 30 minutes, the carrageenan injection or after, e.g., two hours after, the carrageenan injection. Paw pressure sensitivity for each animal is tested with an analgesymeter three hours after the carrageenan injection. See, Randall et al., Arch. Int. Pharmacodyn. (1957) 111:409-419.

The effects of selected agents, e.g., a MIF inhibitor, on carrageenan-induced paw edema can also be examined. This test (see, Vinegar et al., J. Phamacol. Exp. Ther. (1969) 166:96-103) allows an assessment of the ability of a compound to reverse or prevent the formation of edema evoked by paw carrageenan injection. The paw edema test is carried out using a plethysmometer for paw measurements. After administration of a selected agent, a carrageenan solution is injected subcutaneously into the lateral foot pad on the plantar surface of the left hind paw. At three hours post-carrageenan treatment, the volume of the treated paw (left) and the un-treated paw (right) is measured using a plethysmometer.

Von Frey Filament Test: The effect of a MIF inhibitor on mechanical allodynia can be determined by the von Frey filament test in rats with a tight ligation of the L-5 spinal nerve: a model of painful peripheral neuropathy. The surgical procedure is performed as described by Kim et al., Pain (1992) 50 :355-363. A calibrated series of von Frey filaments are used to assess mechanical allodynia (Chaplan et al., J. Neurosci. Methods (1994) 53:55-63). Filaments of increasing stiffness are applied perpendicular to the midplantar surface in the sciatic nerve distribution of the left hindpaw. The filaments are slowly depressed until bending occurred and are then held for 4-6 seconds. The filament application order and number of trials were determined by the up-down method of Dixon (Chaplan et al., supra). Flinching and licking of the paw and paw withdrawal on the ligated side are considered positive responses.

Chronic Constriction Injury: Heat and cold allodynia responses as well as mechanical allodynia sensations can be evaluated as described below in rats having a chronic constriction injury (CCI). A unilateral mononeuropathy is produced in rats using the chronic constriction injury model described in Bennett et al., Pain (1988) 33:87-107. CCI is produced in anesthetized rats as follows. The lateral aspect of each rat's hind limb is shaved and scrubbed with Nolvasan. Using aseptic techniques, an incision is made on the lateral aspect of the hind limb at the mid-thigh level. The biceps femoris is bluntly dissected to expose the sciatic nerve. On the right hind limb of each rat, four loosely tied ligatures (for example, Chromic gut 4.0; Ethicon, Johnson and Johnson, Somerville, N.J.) are made around the sciatic nerve approximately 1-2 mm apart. On the left side of each rat, an identical dissection is performed except that the sciatic nerve is not ligated (sham). The muscle is closed with a continuous suture pattern with, e.g., 4-0 Vicryl (Johnson and Johnson, Somerville, N.J.) and the overlying skin is closed with wound clips. The rats are ear-tagged for identification purposes and returned to animal housing.

Chung Model of Rat Neuropathic Pain: Heat and cold allodynia responses as well as mechanical allodynia sensations can be evaluated as described below in rats following spinal nerve injury (e.g. ligation, transaction). Details are as initially described in S H Kim and J M Chung, Pain (1992) 50:355-363.

The Hargreaves Test: The Hargreaves test (Hargreaves et al., Pain (1998) 32:77-88) is also a radiant heat model for pain. CCI rats are tested for thermal hyperalgesia at least 10 days post-op. The test apparatus consists of an elevated heated (80-82° F.) glass platform. Eight rats at a time, representing all testing groups, are confined individually in inverted plastic cages on the glass floor of the platform at least 15 minutes before testing. A radiant heat source placed underneath the glass is aimed at the plantar hind paw of each rat. The application of heat is continued until the paw is withdrawn (withdrawal latency) or the time elapsed is 20 seconds. This trial is also applied to the sham operated leg. Two to four trials are conducted on each paw, alternately, with at least 5 minutes interval between trials. The average of these values represents the withdrawal latency.

Cold Allodynia Model: The test apparatus and methods of behavioral testing is described in Gogas et al., Analgesia (1997) 3:111-118. The apparatus for testing cold allodynia in neuropathic (CCI) rats consists of a Plexiglas chamber with a metal plate 6 cm from the bottom of the chamber. The chamber is filled with ice and water to a depth of 2.5 cm above the metal plate, with the temperature of the bath maintained at 0-4° C. throughout the test. Each rat is placed into the chamber individually, a timer started, and the animal's response latency was measured to the nearest tenth of a second. A “response” is defined as a rapid withdrawal of the right ligated hindpaw completely out of the water when the animal is stationary and not pivoting. An exaggerated limp while the animal is walking and turning is not scored as a response. The animals' baseline scores for withdrawal of the ligated leg from the water typically range from 7-13 seconds. The maximum immersion time is 20 seconds with a 20-minute interval between trials.

Additional information regarding models of neuropathic pain useful in assessing the MIF inhibitors of the invention is available in the following publications. Bennett G J, Xie Y K (1988) “A peripheral mononeuropathy in rat that produces disorders of pain sensation like those seen in man” Pain 33: 87-107; Chaplan S R, Bach F W, Pogrel J W, Chung J M, Yaksh T L (1994) “Quantitative assessment of tactile allodynia in the rat paw” J. Neurosci. Meth. 53: 55-63; Fox A, Gentry C, Patel S, Kesingland A, Bevan S (2003) “Comparative activity of the anti-convulsants oxcarbazepine, carbamazepine, lamotrigin and gabapentin in a model of neuropathic pain in the rat and guinea-pig” Pain 105: 355-362; Milligan ED, Mehmert K K, Hinde J L, Harvey L O J, Martin D, Tracey K J, Maier S F, Watkins L R (2000) “Thermal hyperalgesia and mechanical allodynia produced by intrathecal administration of the Human Immunodeficiency Virus-1 (HIV-1) envelope glycoprotein, gpl20” Brain Res. 861: 105-116; De Vry J, Kuhl E, Franken-Kunkel P, Eckel G (2004) “Pharmacological characterization of the chronic constriction injury model of neuropathic pain” Eur. J. Pharmacol. 491:137-148. Polomano R C, Mannes A J, Clark U S, Bennett G J (2001) “A painful peripheral neuropathy in the rat produced by the chemotherapeutic drug, paclitaxel” Pain 94:293-304.

Formulations of the Invention

In addition to comprising a MIF inhibitor in accordance with structure I, a therapeutic formulation of the invention may optionally contain one or more additional components as described below.

Excipients/Carriers

In addition to a MIF inhibitor, the compositions of the invention for treating neuropathic pain may further comprise one or more pharmaceutically acceptable excipients or carriers. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

An MIF inhibitor composition of the invention may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition of the invention may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition of the invention may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in a MIF inhibitor composition of the invention is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

Other Actives

A described above, formulation (or kit) in accordance with the invention may contain, in addition to a MIF inhibitor, one or more additional active agents effective in treating neuropathic pain. Such actives include ibudilast, gabapentin, memantine, pregabalin, morphine and related opiates, cannabinoids, tramadol, lamotrigine, carbamazepine, duloxetine, milnacipran, and tricyclic antidepressants.

Gabapentin, also known as Neurontin®, is structurally related to the neurotransmitter GABA. Although structurally related to GABA, gabapentin does not interact with GABA receptors, is not converted metabolically into GABA or a GABA agonist, and is not an inhibitor of GABA uptake or degradation. Gabapentin has no activity at GABAA or GABAB receptors of GABA uptake carriers of the brain, but instead interacts with a high-affinity binding site in brain membranes (an auxiliary subunit of voltage-sensitive Ca²⁺ channels). The exact mechanism of action is unknown, only that its physiological site of action is the brain. The structure of gabapentin allows it to pass freely through the blood-brain barrier. In vitro, gabapentin has many pharmacological actions including modulating the action of the GABA synthetic enzyme, increasing non-synaptic GABA responses from neural tissue, and reduction of the release of several mono-amine neurotransmitters. Daily dosages of gabapentin typically range from about 600 to 2400 mg/day, more preferably from about 900 to 1800 mg/day, and are administered in divided doses, for example, three times a day. Conventional unit dosage forms are 300 or 400 mg capsules or 600 or 800 mg tablets.

The active agent, memantine, is a receptor antagonist. Memantine is believed to function as a low to moderate affinity uncompetitive (open-channel) NMDA receptor antagonist which binds to the NMDA receptor-operated cation channels. Recommended daily dosage amounts typically range from about 5 mg to 20 mg.

The opiate, morphine, elicits its effects by activating opiate receptors that are widely distributed throughout the brain and body. Once an opiate reaches the brain, it quickly activates the opiate receptors found in many brain regions and produces an effect that correlates with the area of the brain involved. There are several types of opiate receptors, including the delta, mu, and kappa receptors. Opiates and endorphins function to block pain signals by binding to the mu receptor site.

The cannabinoids, e.g., tetrahydrocannabinol, bind to the cannabinoid receptor referred to as CB₁. CB₁ receptors are found in brain and peripheral tissues; CB₁ receptors are present in high quantities in the central nervous system, exceeding the levels of almost all neurotransmitter receptors. An additional cannabinoid receptor subtype termed ‘CB2’ has also been identified. See, e.g., Martin, B. R., et al., The Journal of supportive Oncology, Vol. 2, Number 4, July/August 2004.

Although its mechanism of action has not yet been fully elucidated, the opioid, tramadol, is believed to work through modulation of the GABAergic, noradrenergic and serotonergic systems. Tramadol, and its metabolite, known as M1, have been found to bind to μ-opioid receptors (thus exerting its effect on GABAergic transmission), and to inhibit re-uptake of 5-HT and noradrenaline. The second mechanism is believed to contribute since the analgesic effects of tramadol are not fully antagonised by the μ-opioid receptor antagonist naloxone. Typical daily dosages range from about 50 to 100 milligrams every 4 to 6 hours, with a total daily dosage not to exceed 400 milligrams.

Lamotrigine is a phenyltriazine that stabilizes neuronal membranes by blocking voltage-sensitive sodium channels, which inhibit glutamate and aspartate (excitatory amino acid neurotransmitter) release. The daily dosage of lamotrigine typically ranges from 25 milligrams per day to 500 mg per day. Typical daily dosage amounts include 50 mg per day, 100 mg per day, 150 mg per day, 200 mg per day, 300 mg per day, and 500 mgs per day, not exceed 700 mgs per day.

Carbamazepine acts by blocking voltage-sensitive sodium channels. Typical adult dosage amounts range from 100-200 milligrams one or two times daily, to an increased dosage of 800-1200 milligrams daily generally administered in 2-3 divided doses.

Duloxetine is a potent inhibitor of neuronal uptake of serotonin and norephinephrine and a weak inhibitor of dopamine re-uptake. Typical daily dosage amounts range from about 40 to 60 milligrams once daily, or 20 to 30 milligrams twice daily.

Milnacipran acts as a serotonin and norepinephrine reuptake inhibitor. Daily dosage amounts typically range from about 50 to 100 milligrams once or twice daily.

The dosage amounts provided above are meant to be merely guidelines; the precise amount of a secondary active agent to be administered during combination therapy with a MIF inhibitor will, of course, be adjusted accordingly and will depend upon factors such as intended patient population, the particular neuropathic pain symptom or condition to be treated, potential synergies between the active agents administered, and the like, and will readily be determined by one skilled in the art based upon the guidance provided herein.

Sustained Delivery Formulations

The compositions may also be formulated in order to improve stability and extend the half-life of the MIF inhibitor. For example, the MIF inhibitor may be delivered in a sustained-release formulation. Controlled or sustained-release formulations are prepared by incorporating the MIF inhibitor into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, the MIF inhibitor can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

Delivery Forms

The MIF inhibitor compositions described herein encompass all types of formulations, and in particular, those that are suited for systemic or intrathecal administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned. Preferably, a MIF inhibitor composition of the invention is one suited for oral administration.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Formulations for topical administration in the mouth include lozenges comprising the active ingredients, generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil.

Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate.

Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERx™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations of the invention are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation of the invention may also be a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents.

The compositions of the present invention may also be prepared in a form suitable for veterinary applications.

Kits

Also provided herein is a kit containing a MIF inhibitor composition of the invention, accompanied by instructions for use, e.g., in treating neuropathic pain. The packaging may be in any form commonly employed for the packaging of pharmaceuticals, and may utilize any of a number of features such as different colors, wrapping, tamper-resistant packaging, blister packs, dessicants, and the like.

Methods for Treating Addictions

Another aspect of the present invention relates to a therapeutic methodology for safely and effectively treating addiction by administering one or more of the quinoline or 2-quinolone based MIF inhibitor compounds described herein. The methods provided herein are thought to reduce the release of dopamine in the nucleus accumbens, which is associated with cravings and compulsive behavior in addicts. The methods of the invention are particularly useful in diminishing or eliminating addiction-related behavior and alleviating symptoms of withdrawal syndromes in a subject.

In arriving at this aspect of the invention, the inventors realized that in developing an improved treatment for opioid withdrawal, it is important to consider that opioids, including morphine, do not just affect neurons. While opioid-responsive neurons in various brain and spinal cord regions suppress pain, lower core body temperature, alter hormone release, etc. (the classical effects of opioids), it has recently been discovered that opioids also affect a non-neuronal cell type called glia (microglia, astrocytes, oligodendrocytes). Morphine and other opioids activate glia. This activation increases with repeated opioid administration, as evidenced by the upregulation of glia-specific activation markers. That such glial activation contributes to morphine tolerance is supported by the finding that co-administering glial inhibitors along with morphine disrupts the development of morphine tolerance. The inventors recognized that reduction of glial activation may be useful as a therapeutic approach to disrupting the development of morphine tolerance. Watkins, L. R. et al. (2005) Trends in Neuroscience 28:661-669; Gul, H. et al. (2000) Pain 89:39-45; Johnston, I. N. et al. (2004) J. Neurosci. 24:7353-65; Raghavendra, V. et al. (2002) J. Neurosci 22 (22):9980-89; Raghavendra, V. et al. (2004) Neuropsychopharmacology 29 (2):327-34; Shavit, Y. et al. (2005) Pain 115:50-59; Song, P. and Zhao, Z. Q. (2001) Neurosci. Res. 39:281-86. Moreover, it is the inventors' belief that since ibudilast has been shown to function as both as glial attenuator and a MIF inhibitor, that such action can highly likely be attributed to the quinoline and 2-quinolone based MIF inhibors described herein.

Opioid-driven progressive glial activation causes glia to release neuroexcitatory substances, including the proinflammatory cytokines interleukin-1 (IL-1), tumor necrosis factor (TNF), and interleukin-6 (IL-6). These neuroexcitatory substances counteract the pain-relieving actions of opioids, such as morphine, and drive withdrawal symptomology, as demonstrated by experiments involving co-administration or pro- or anti-inflammatory substances along with morphine. For example, injecting IL-1 into the cerebrospinal fluid of mice at a dose having no behavioral effect on its own blocks the analgesic effect of systemic morphine. Similarly, spinal delivery of morphine and IL-1 receptor antagonist (which prevents IL-1 from exerting its effects), or morphine and the anti-inflammatory cytokine IL-10 (which downregulates the production, release and efficacy of proinflammatory cytokines), enhances the magnitude and duration of morphine analgesia. Indeed, if morphine analgesia is established and then allowed to dissipate, potent analgesia can be rapidly reinstated by injecting IL-1 receptor antagonist, suggesting that dissipation of analgesia is caused by the activities of pain-enhancing proinflammatory cytokines rather than dissipation of morphine's analgesic effects.

The activity of other opioids may also be opposed by activation of glia. Studies show that glia and proinflammatory cytokines compromise the analgesic effects of methadone, at least in part, via non-classical opioid receptors (Watkins, L. R. et al. (2005) Trends Neurosci. 28:661-669). These results suggest that glia and proinflammatory cytokines will be involved in methadone withdrawal, and likely withdrawal from other opioids as well. These data also expand the clinical implications of glial activation, since cross-tolerance between opioids may be explained by the activation of the glial pain facilitatory system, which undermines all attempts to treat chronic pain with opioids.

In summary, opioids excite glia, which in turn release neuroexcitatory substances (such as proinflammatory cytokines) that oppose the effects of opioids and create withdrawal symptoms upon cessation of opioid treatment. Compounds that suppress such glial activation (e.g., the MIF inhibitors described herein) would be beneficial novel therapeutics for treatment of opioid withdrawal and related applications.

Treatment of Addictions with Quinoline or 2-Ouinolone Based MIF Inhibitor Compounds

Dopamine release in the nucleus accumbens is thought to mediate the “reward” motivating drug use and compulsive behavior associated with addictions. In one aspect, the invention provides a method for suppressing the release of dopamine in the nucleus accumbens of a subject comprising administering to the subject a composition comprising an effective amount of a MIF inhibitor corresponding to structure I (described previously).

Thus, the MIF inhibitors described generally by structure I (including exemplary structures II-VII) are useful in the treatment of addictions, and in particular, are useful in attenuating or abolishing the dopamine mediated “reward” associated with addictions, thus diminishing or eliminating cravings associated with addictions and the accompanying addiction-related behavior and withdrawal syndromes of a subject.

One method of the invention includes administering a therapeutically effective amount of a MIF inhibitor as described herein to a subject for treating a drug addiction. The subject may be addicted to one or more classifications of drugs including, but not limited to, psychostimulants, narcotic analgesics, alcohols and addictive alkaloids, such as nicotine, cannabinoids, or combinations thereof.

Examples of psychostimulants include, but are not limited to, amphetamine, dextroamphetamine, methamphetamine, phenmetrazine, diethylpropion, methylphenidate, cocaine, phencyclidine, methylenedioxymethamphetamine and pharmaceutically acceptable salts thereof.

Narcotic analgesics include, but are not limited to, alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine and pharmaceutically acceptable salts thereof.

Addictive drugs also include central nervous system depressants, including, but not limited to, barbiturates, chlordiazepoxide, and alcohols, such as ethanol, methanol, and isopropyl alcohol.

A quinoline or 2-quinolone-based MIF inhibitor may also be administered to a subject to treat a behavioral addiction, e.g., compulsive eating, drinking, smoking, shopping, gambling, sex, and computer use.

In certain embodiments, such MIF-inhibitors are used in combination with one or more other agents for treating an addiction. Such agents include, but are not limited to, the following classes of drugs: analgesics, NSAIDs, antiemetics, antidiarrheals, alpha-2-antagonists, benzodiazepines, anticonvulsants, antidepressants, and insomnia therapeutics. Such agents include, but are not limited to, buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfiram, acamprosate, topiramate, ondansetron, sertraline, bupropion, amantadine, amiloride, isradipine, tiagabine, baclofen, propranolol, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, naltrexone, paracetamol, metoclopramide, loperamide, clonidine, lofexidine, and diazepam.

Treatment of Opiate Withdrawl

The present invention also relates to approaches for treating opioid dependence and withdrawal, and specifically the use of a MIF inhibitor as described herein as an effective therapeutic treatment for morphine withdrawal. The clinical manifestations of morphine withdrawal are thought to result, in part, from glial activation in the central nervous system (Narita et al. (2006) Nature Neuropsychopharmacology 1-13), and, it is the inventors' view that the subject MIF inhibitors possess the ability to down-regulate glial cell activation. Thus, systemic (e.g. oral) or central (e.g. intrathecal) administration of such MIF inhibitors provides a novel approach to attenuate morphine withdrawal, thereby providing an effective treatment for a condition with few good therapeutic options.

A growing body of literature suggests that repetitive morphine treatment may result in glial cell (microglia, astrocytes) activation, and that such activation may contribute to the sequelae of events associated with morphine tolerance and withdrawal.

Several cues activate glia, such as immune challenges, infection and/or peripheral inflammation, substances released during prolonged neuron-to-neuron transmission (e.g., neurotransmitters, nitric oxide, prostaglandins, substance P, fractalkine, etc.), neuronal damage (e.g., fractalkine, heat shock proteins, cell wall components), etc. Glial function is changed dramatically upon activation, resulting in elevated release of neuroactive substances. Such events are thought to contribute to altered neurological function with manifestations ranging from neurodegeneration, to pain facilitation, to sensitization of morphine dependence and subsequent withdrawal syndrome. Watkins and Maier (2002) Physiol. Rev. 82: 981-1011; Watkins and Maier (2004) Drug Disc. Today: Ther. Strategies 1(1): 83-88, etc.

According to the present invention, the subject MIF inhibitors can be used to reduce such undesired glial activation. While certain agents like minocycline and fluorocitrate may have some activity preventing glial activation, they are unacceptable for human therapy. Fluorocitrate is unacceptable because it can block glial uptake of excitatory amino acids (Berg-Johnsen et al. (1993) Exp. Brain Res. 96(2):241-6), an essential function of glia in the maintenance of normal CNS homeostasis, and extended duration or increased doses of fluorocitrate cause seizures. Willoughby J. O., et al. (2003) J. Neurosci. Res. 74(1):160-66; Homfeldt, C. S. and Larson, A. A. (1990) Eur. J. Pharmacol. 179(3):307-13. While minocycline may be useful in preventing glial activation, it does not appear to be able to reverse extant situations. Raghavendra et al. (2003) J. Pharmacol. and Exp. Therapeutics 306: 624-30; Ledeboer, A., et al. (2005) Pain 115:71-83.

Taken together, glia and their pro-inflammatory or neuromodulatory products present opportunities for new strategies for control of morphine withdrawal by administration of the subject MIF inhibitors.

A MIF inhibitor as described herein may also be administered in combination with one or more other agents as part of a comprehensive opioid withdrawal treatment protocol. Such agents include, but are not limited to, the following: naltrexone, metoclopramide, loperamide, diazepam, clonidine, and paracetemol.

It is to be understood that while the invention has been described in conjunction with preferred specific embodiment, the foregoing description is intended to illustrate and not limit the scope of the invention. Other aspects, advantages, and modifications within the scope of the invention will be apparent to those skilled in the art to which the invention pertains.

1. A method of treating a mammalian subject suffering from neuropathic pain, said method comprising:
administering to said subject a therapeutically effective amount of a MIF inhibitor having the following structure: 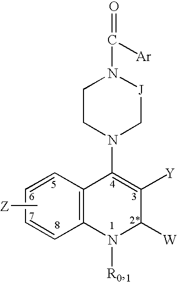
where
Ar is aryl, substituted aryl, heteroaryl, or substituted heteroaryl,
J is lower alkylene or carbonyl (C═O),
Y is —NO₂, or CO₂R₂
R₀ indicates the absence of a substituent on N1,
R₁ indicates the presence of a substituent on N1 selected from hydrogen, alkyl, —(CH₂)ₙAr, and (CH₂)ₙN(CH₃)₂ where n ranges from 1-5,
R₂ is lower alkyl,
Z is —H, halo, or lower alkyl, and,
when N₁ is attached to R₁, W, when taken together with C₂, forms a carbonyl (═O), and
when N₁ is attached to R₀, then N₁ forms a double bond with C₂, and W is either —OR₂ or —SR₂,
whereby as a result of said administering, the subject experiences relief of said neuropathic pain.

2. The method of claim 1, wherein the subject suffers from postherpetic neuralgia, trigeminal neuralgia, or neuropathic pain associated with a condition selected from the group consisting of herpes, HIV, traumatic nerve injury, stroke, post-ischemia, fibromyalgia, reflex sympathetic dystrophy, complex regional pain syndrome, spinal cord injury, sciatica, phantom limb pain, multiple sclerosis, and cancer chemotherapeutic-induced neuropathic pain.

3. The method of claim 1, wherein said administering comprises systemically administering said MIF inhibitor.

4. The method of claim 3, wherein said MIF inhibitor is administered by oral, intravenous, subcutaneous, intramuscular, intraperitoneal, intranasal, or sublingual adminstration.

5. The method of claim 1, wherein said administering comprises centrally administering said MIF inhibitor by intrathecal, intraspinal or intranasal administration.

6. The method of claim 1, wherein said administering comprises once daily dosing.

7. The method of claim 1, wherein said administering comprises twice or thrice daily dosing.

8. The method of claim 1, wherein said administering is over a time course of at least about a week.

9. The method of claim 1, wherein said administering is over a time course ranging from about one week to 50 weeks.

10. The method of claim 1, whereby said subject is experiencing allodynia, and said administering is effective to relieve allodynia experienced by said subject.

11. The method of claim 1, wherein said administering is effective to attenuate neuropathic pain experienced by said subject for up to at least 8 hours post MIF-inhibitor administration.

12. The method of claim 1, wherein said administering comprises administering said MIF inhibitor in combination with an additional agent effective for treating neuropathic pain.

13. The method of claim 12, wherein said additional agent is ibudilast, gabapentin, memantine, pregabalin, morphine and related opiates, cannabinoids, tramadol, lamotrigine, lidocaine, carbamazepine, duloxetine, milnacipran, and/or tricyclic antidepressants.

14. The method of claim 1, wherein Ar is thiophene or pyrrole.

15. The method of claim 1, wherein Z is positioned at the 6-position of the quinoline or 2-quinolone ring structure.

16. The method of claim 15, wherein Z is hydrogen, chloro, or methyl.

17. The method of claim 1, where W, when taken together with C₂, forms a carbonyl.

18. The method of claim 1, where W is either —OR₂ or —SR₂ and N₁ forms a double bond with C₂.

19. The method of claim 1, wherein the MIF inhibitor possesses a structure selected from: 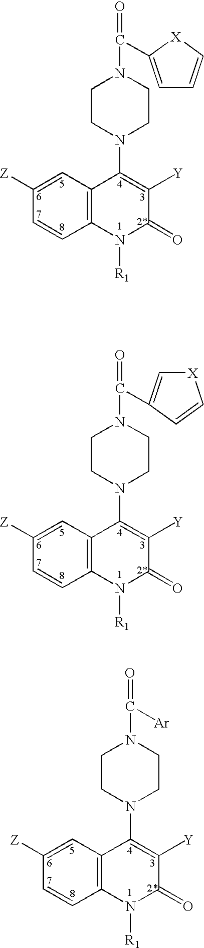
and X is sulfur or oxygen.

20. The method of claim 19, wherein
Z is hydrogen, chloro, or methyl, and
Y is —NO₂, —C(O)OCH₃, —C(O)OCH₂CH₃, —C(O)OCH₂CH₂CH₃, or —C(O)OCH(CH₃)₂.

21. The method of claim 1, wherein the MIF inhibitor possesses a structure selected from: 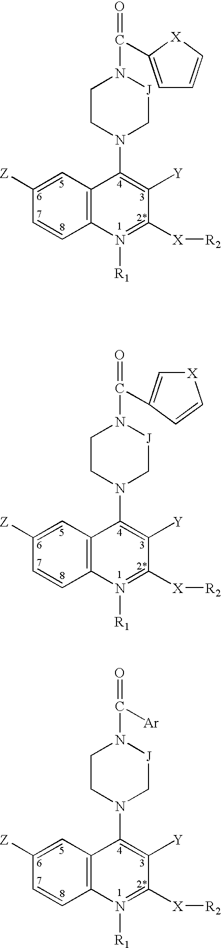
and where X is oxygen or sulfur.

22. The method of claim 21, wherein:
Z is hydrogen, chloro, or methyl, and
Y is —NO₂, —C(O)OCH₃, —C(O)OCH₂CH₃, —C(O)OCH₂CH₂CH₃, or —C(O)OCH(CH₃)₂.
